(19) United States
(12) Patent Application Publication
(10) Pub. No.: US 2003/0017451 A1
(43) Pub. Date: Jan. 23, 2003

(54) Methods for detecting transcripts

(76) Inventors: Hui Wang, San Jose, CA (US); Steven Smeekens, Belmont, CA (US); Glenn H. McGall, Mountain View, CA (US); Yanxiang Cao, Mountain View, CA (US)

Correspondence Address: Wei Zhou, Affymetrix, Inc., 3380 Central Expressway, Santa Clara, CA, 95051, US

(21) Appl. No.: 09/746,113

(22) Filed: Dec. 21, 2000

(51) Int. Cl. C12Q 1/68

(52) U.S. Cl. 435/6

(57) ABSTRACT

Methods are provided for analyzing a large number of RNAs. In one embodiment of the invention, RNAs are hybridized with a nucleic acid probe (primer) array. The primers are extended using reverse transcriptase with the RNAs as templated. The resulting primer extension products are detected. The methods are useful for gene expression monitoring, sequence variation detection and transcriptional mapping/annotation.

### TECHNICAL FIELD

The present invention is in the field of genetic analysis for medical diagnosis, genetic variation research, or genetic engineering. More specifically, the present invention is in the field of nucleic acid analysis.

### BACKGROUND

Many cellular events and processes are characterized by altered expression levels of one or more genes. Differences in gene expression correlate with many physiological processes such as cell cycle progression, cell differentiation and cell death. Changes in gene expression patterns also correlate with changes in disease or pharmacological state. For example, the lack of sufficient expression of functional tumor suppressor genes and/or the over expression of oncogene/protooncogenes could lead to tumorgenesis (Marshall, Cell, 64: 313-326 (1991); Weinberg, Science, 254: 1138-1146 (1991), incorporated herein by reference in their entireties for all purposes). Thus, changes in the expression levels of particular genes (e.g. oncogenes or tumor suppressors) serve as signposts for different physiological, pharmacological and disease states.

Recently, massive parallel gene expression monitoring methods have been developed to monitor the expression of a large number of genes using nucleic acid array technology which was described in detail in, for example, U.S. Pat. No. 5,871,928; de Saizieu, et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference in their entireties for all purposes.

However, there is still great need in the art for additional methods for monitoring the expression of a large number of genes.

### SUMMARY OF THE INVENTION

In one aspect of the invention, methods that are useful for interrogating the entire sequence of RNA molecules are provided. The methods include hybridizing the sample with a substrate, where the substrate has a plurality of probes and wherein the probes are suitable for primer extension reactions; synthesizing primer extension products with a nucleic acid polymerase, appropriate reagents and conditions, from the primers and using the RNAs as templates; and detecting the primer extension products. In preferred embodiments, the nucleic acid polymerase is a reverse transcriptase. In some embodiments, the reverse transcriptase is a thermostable reverse transcriptase.

In preferred embodiments, the probes are oligonucleotide probes, preferrably immobilized on the substrate in 5′-3′ direction. The oligonucleotides can be synthesized on the substrated in 5′-3′ direction or alternatively spotted on the substrate. Photodirected synthesis is preferred for high density arrays. Other methods of synthesis, such as mechanic channels, ink-jet printer like reagent delivery systems can also be used. The substrate preferably has at least 100, 1000, 1000 probes per per cm² of the substrate.

The methods are particularly useful for analyzing a large number of, at least 50, 100, 1000, 5000, RNAs simutaneously. In preferred embodiments, each of the RNAs is targeted by at least 2, 5, 10 or 20 probes.

The extension products may be detected by using a label, such as a fluorescence label. The label may be incorporated during the synthesizing or attached to the extension products after the synthesizing.

In some embodiments, the probes include tiling probes that are selected to tile regions of the RNA, and the methods further include a step of determining sequence variations by detecting the extension products of the tiling probes. The sequence variations may be SNPs.

In some other embodiments, the probes include tiling probes that are selected to tile the bordering regions of exons or putative exons, and the methods further include determining the arrangement of exons in the RNAs by detecting the extension products of the tiling probes.

In some additional embodiments, the probes include probes that are designed to target subregions of a genomic sequence and the methods further include determining whether the subregions of the genomic sequence is transcribed by detecting the extension products of the probes designed to target the subregions of the genomic sequence.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic illustrating one embodiment of the invention.

FIG. 2 shows the synthesis of 5′-DMT-3′-MeNPOC-Nucleosides.

FIG. 3 shows the synthesis of 3′-MeNPOC-Nucleosides (10 g scale).

FIG. 4 shows the synthesis of 5′-MeNPOC-2′-Deoxynucleoside-3-(2-cyanoetyl-N,N-Diisopropylphosphoramidites.

FIG. 5 shows relative hybridization signal intensity of 3′-5′ and 5′->3′ synthesis efficiency.

FIG. 6 shows an image of high density oligonucleotide probe arrays with extension products.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

I. General

The present invention relies on many patents, applications and other references for certain details known to those of the art. Therefore, when a patent, application, or other reference is cited or repeated below, it should be understood that it is incorporated by reference in its entirety for all purposes as well as for the proposition that is recited.

As used in the specification and claims, the singular form “a” “an,” and “the”include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, detection of hybridization using a label. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

Additional methods and techniques applicable to array synthesis have been described in U.S. Pat. Nos. 5,143,854, 5,242,974, 5,252,743, 5,324,633, 5,384,261, 5,405,783, 5,412,087, 5,424,186, 5,445,934, 5,451,683, 5,482,867, 5,489,678, 5,491,074, 5,510,270, 5,527,681, 5,550,215, 5,571,639, 5,578,832, 5,593,839, 5,599,695, 5,624,711, 5,631,734, 5,677,195, 5,744,101, 5,744,305, 5,770,456, 5,795,716, 5,800,992, 5,831,070, 5,837,832, 5,856,101, 5,871,928, 5,858,659, 5,936,324, 5,968,740, 5,974,164, 5,981,185, 5,981,956, 6,025,601, 6,033,860, 6,040,138, and 6,090,555, which are all incorporated herein by reference in their entirety for all purposes.

Analogue when used in conjunction with a biomonomer or a biopolymer refers to natural and un-natural variants of the particular biomonomer or biopolymer. For example, a nucleotide analogue includes inosine and dideoxynucleotides. A nucleic acid analogue includes peptide nucleic acids. The foregoing is not intended to be exhaustive but rather representative. More information can be found in U.S. patent application Ser. No. 80/630,427.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementarity. See e. g., M. Kanehisa Nucleic Acids Res. 12:203 (1984), incorporated herein by reference.

Hybridization refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25EC. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

Nucleic acid refers to a polymeric form of nucleotides of any length, such as oligonucleotides or polynucleotides, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be customized to stabilize or destabilize hybrid formation or enhance the specificity of hybridization with a complementary nucleic acid sequence as desired.

Oligonucleotide or polynucleotide is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

Primer is a single-stranded oligonucleotide capable of acting as a point of initiation for template-directed DNA synthesis under suitable conditions, e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 3 to 6 and up to 30 or 50 nucleotides. Short primer molecules generally require cooler temperatures to form sufficiently stable hybrid complexes with the template. A primer needs not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Single Nucleotide Polymorphism or SNP occurs at a polymorphic site occupied by a single nucleotide, which is the site of variation between allelic sequences. This site of variation is usually both preceded by and followed by highly conserved sequences e.g., sequences that vary in less than 1/100 or 1/1000 members of the populations of the given allele. A SNP usually arises due to the substitution of one nucleotide for another at the polymorphic site. These substitutions include both transitions (i.e. the replacement of one purine by another purine or one pyrimidine by another pyrimidine) and transversions (i.e. the replacement of a purine by a pyrimidine or vice versa). SNPs can also arise from either a deletion of a nucleotide or from an insertion of a nucleotide relative to a reference allele.

Substrate refers to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

High density nucleic acid probe arrays, also referred to as “DNA Microarrays,” have become a method of choice for monitoring the expression of a large number of genes.

A target molecule refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51. For example, if transcripts of genes are the interest of an experiment, the target molecules would be the transcripts. Other examples include protein fragments, small molecules, etc. Target nucleic acid refers to a nucleic acid (often derived from a biological sample) of interest. Frequently, a target molecule is detected using one or more probes. As used herein, a probe is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that there are illustrative embodiments that are not to limit the invention in any way.

In preferred embodiments, probes may be immobilized on substrates to create an array. An array may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes.

Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996, 6,136,269 and attorney docket numbers 3298.1 and 3309, all of which are incorporated by reference in their entireties for all purposes.

Nucleic acid probe array technology, use of such arrays, analysis array based experiments, associated computer software, composition for making the array and practical applications of the nucleic acid arrays are also disclosed, for example, in the following U.S. patent application Ser. Nos. 07/838,607, 07/883,327, 07/978,940, 08/030,138, 08/082,937, 08/143,312, 08/327,522, 08/376,963, 08/440,742, 08/533,582, 08/643,822, 08/772,376, 09/013,596, 09/016,564, 09/019,882, 09/020,743, 09/030,028, 09/045,547, 09/060,922, 09/063,311, 09/076,575, 09/079,324, 09/086,285, 09/093,947, 09/097,675, 09/102,167, 09/102,986, 09/122,167, 09/122,169, 09/122,216, 09/122,304, 09/122,434, 09/126,645, 09/127,115, 09/132,368, 09/134,758, 09/138,958, 09/146,969, 09/148,210, 09/148,813, 09/170,847, 09/172,190, 09/174,364, 09/199,655, 09/203,677, 09/256,301, 09/285,658, 09/294,293, 09/318,775, 09/326,137, 09/326,374, 09/341,302, 09/354,935, 09/358,664, 09/373,984, 09/377,907, 09/383,986, 09/394,230, 09/396,196, 09/418,044, 09/418,946, 09/420,805, 09/428,350, 09/431,964, 09/445,734, 09/464,350, 09/475,209, 09/502,048, 09/510,643, 09/513,300, 09/516,388, 09/528,414, 09/535,142, 09/544,627, 09/620,780, 09/640,962, 09/641,081, 09/670,510, 09/685,011, and 09/693,204 and in the following Patent Cooperative Treaty (PCT) applications/publications: PCT/NL90/00081, PCT/GB91/00066, PCT/US91/08693, PCT/US91/09226, PCT/US91/09217, WO/93/10161, PCT/US92/10183, PCT/GB93/00147, PCT/US93/01152, WO/93/22680, PCT/US93/04145, PCT/US93/08015, PCT/US94/07106, PCT/US94/12305, PCT/GB95/00542, PCT/US95/07377, PCT/US95/02024, PCT/US96/05480, PCT/US96/11147, PCT/US96/14839, PCT/US96/15606, PCT/US97/01603, PCT/US97/02102, PCT/GB97/005566, PCT/US97/06535, PCT/GB97/01148, PCT/GB97/01258, PCT/US97/08319, PCT/US97/08446, PCT/US97/10365, PCT/US97/17002, PCT/US97/16738, PCT/US97/19665, PCT/US97/20313, PCT/US97/21209, PCT/US97/21782, PCT/US97/23360, PCT/US98/06414, PCT/US98/01206, PCT/GB98/00975, PCT/US98/04280, PCT/US98/04571, PCT/US98/05438, PCT/US98/05451, PCT/US98/12442, PCT/US98/12779, PCT/US98/12930, PCT/US98/13949, PCT/US98/15151, PCT/US98/15469, PCT/US98/15458, PCT/US98/15456, PCT/US98/16971, PCT/US98/16686, PCT/US99/19069, PCT/US98/18873, PCT/US98/18541, PCT/US98/19325, PCT/US98/22966, PCT/US98/26925, PCT/US98/27405 and PCT/IB99/00048, all of which are incorporated by reference in their entireties for all purposes. All the above cited patent applications and other references cited throughout this specification are incorporated herein by reference in their entireties for all purposes.

The embodiments of the invention will be described using GeneChip® high oligonucleotide density probe arrays (available from Affymetrix, Inc., Santa Clara, Calif., USA) as exemplary embodiments. One of skill the art would appreciate that the embodiments of the invention are not limited to high density oligonucleotide probe arrays. In contrast, the embodiments of the invention are useful for analyzing any parallel large scale biological analysis, such as those using nucleic acid probe array, protein arrays, etc.

Gene expression monitoring using GeneChip® high density oligonucleotide probe arrays are described in, for example, Lockhart et al., 1996, Expression Monitoring By Hybridization to High Density Oligonucleotide Arrays, Nature Biotechnology 14:1675-1680; U.S. Pat. Nos. 6,040,138 and 5,800,992, all incorporated herein by reference in their entireties for all purposes.

II. Gene Expression Monitoring by Primer Extension

Many gene expression assays employ in vitro reverse transcription reactions using poly-(dT) primers which hybridizes with the 3′ end of most Eukaryote mRNAs. Because of relative low efficiency of reverse transcription or the amplification of the transcripts using poly(T), it is difficult to interrogate further than 600 bases from the 3′ poly-(A) tail of mRNAs, while the average size of mRNAs is about 1500 bases.

In such assays, probes for detecting their target mRNAs are mostly selected along the regions close to the 3′ poly-(A) tail of mRNAs because a region that is too far away from the 3′ end may not be represented in the nucleic acid samples to be hybridized with the array. As a result, analysis of mRNAs in such assays may be 3′ biased in some analysis systems. This 3′ bias limits the ability to interrogate and quantify biologically relevant information in much of the transcripts.

In addition to expression monitoring, this limitation includes detection of alternatively spliced transcripts, genotyping, and the discovery and detection of SNPs. These limitations will become even more critical as additional sequence information is obtained from the Human Genome Project and understanding the genetic variations among individuals becomes more critical.

In one aspect of the invention, the present invention is directed to solve the 3′ bias problem so that the sequences of whole molecule of mRNA can be interrogated in a simple, fast and reproducible process.

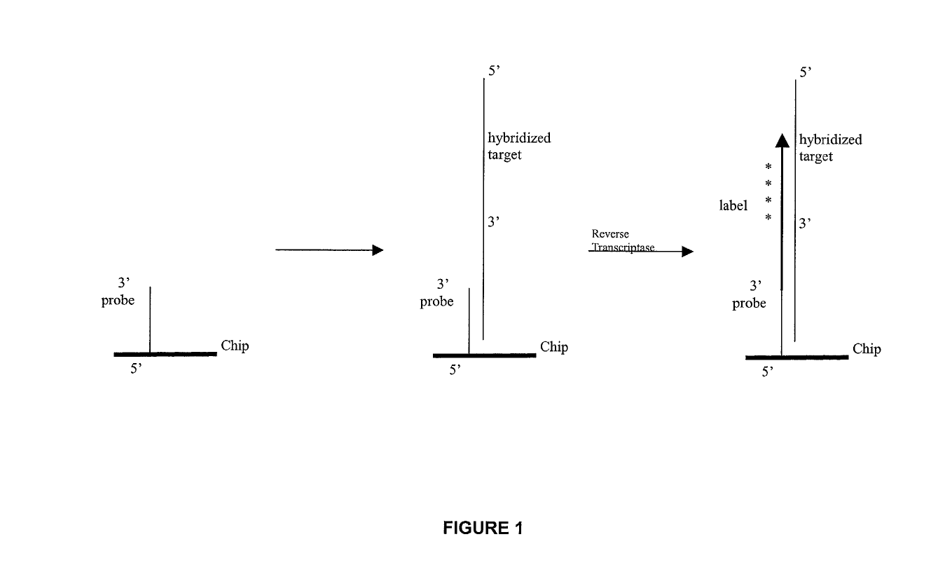

FIG. 1 shows an exemplary method of the invention. Oligonucleotide probes are synthesized on a substrate in a 5′-3′ direction. To capture or interrogate a sequence corresponding to a transcript, an oligonucleotide corresponding to the (−)-strand would be synthesized having the sequence. The RNAs isolated from cells can be captured or interrogated directly since its sequence corresponds to the reverse complement of the probe sequence on the substrate. The captured sequence may be labeled so that it can be detected on the chip. One labeling method employs directly labeling by reverse transcription of the captured target in the presence of biotin-labeled nucleotides. The biotin labeled newly synthesized sequence may be detected using, e.g., fluorescence labeling and scanning. One of skill in the art would appreciate that the probe arrays that are synthesized in 5′-3′ direction may also be used for hybridization based gene expression analysis.

Some preferred embodiments of the invention include providing a plurality of probes having complimentary sequences to specific RNAs, about 10, 15, 20, 25, 30, 35, 40, 45, 50 bases each, on a solid substrate. The probes also serve as primers and therefore, the immobilization of the probes is compatible for their function as primer. In exemplary embodiments, the probes may be synthesized from the substrate in 5′-3′ direction.

A RNA sample is hybridized with the immobilized primers under appropriate conditions. Complementary DNA (cDNA) molecules are synthesized from the primer-RNA complex with a reverse transcriptase, with appropriate reagents and conditions. The synthesized cDNA (primer extension products) may be labeled and detected.

The methods of the invention are not only suitable for detecting mammalian mRNAs with poly(A) tail. Instead, the methods of the invention are useful for detecting any type of RNAs including but not limited to nascent and processed mRNAs, prokaryote mRNAs, tRNA, small RNAs.

In a particularly preferred embodiments, for each RNA molecule, there are a number of, preferably at least 2, 5, 10, 15, 20, 25, 30, 35, or 40 probes (primers), for detecting a single RNA molecule. The target regions of the probes in such embodiments may be distributed along the target RNA molecules. Therefore, the methods of the invention are not subject to the 3′ bias problem resulting from the use of poly(dT) primers in in vitro reverse transcription reaction. In some preferred embodiments, the probes (primers) are selected to cover the entire sequence of RNA molecules. In such embodiments, the entire RNA sequence may be interrogate, not only for quantitative information, but also sequence variations including alternatively arranged exons, mutations and polymorphisms.

III. Sample Preparation

The methods of the invention are not limited to any particular method of sample preparation. A large number of well-known methods for isolating and purifying RNA are suitable for this invention.

One of skill in the art will appreciate that it is desirable to have nucleic samples containing target nucleic acid sequences that reflect the transcripts of interest. Therefore, suitable nucleic acid samples may contain transcripts of interest. Suitable nucleic acid samples, however, may also contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Transcripts, as used herein, may include, but not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

In one embodiment, such a sample is a homogenate of cells or tissues or other biological samples. Preferably, such sample is a total RNA preparation of a biological sample. More preferably in some embodiments, such a nucleic acid sample is the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with poly (T) column contains RNA molecules with poly (A) tails. Those poly A+ RNA molecules could be mature mRNA, RNA processing intermediates, nascent transcripts or degradation intermediates.

Biological samples may be of any biological tissue or fluid or cells. Frequently the sample will be a “clinical sample” which is a sample derived from a patient. Clinical samples provide a rich source of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Another typical source of biological samples are cell cultures where gene expression states can be manipulated to explore the relationship among genes. In one aspect of the invention, methods are provided to generate biological samples reflecting a wide variety of states of the genetic network.

One of skill in the art would appreciate that it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In some other embodiments, RNase are inhibited or destroyed by heart treatment followed by proteinase treatment.

Methods of isolating total RNA and mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In a preferred embodiment, the total RNA is isolated from a given sample using, for example, an acid guanidinium-phenol-chloroform extraction method and polyA⁺ mRNA is isolated by oligo (dT) column chromatography or by using (dT) magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Most of eukaroytic mRNA have 3′ poly (A) tails, some of eukaroytic and all of prokaroytic mRNA do not contain 3′ poly (A) tails. While the methods of the invention does not rely upon the 3′ poly (A) tails of eukaroytic mRNA mRNAs for cDNA synthesis and amplification, it is still often desirable to isolate mRNAs from RNA samples.

In one particularly preferred embodiment, total RNA is isolated from mammalian cells using RNeasy Total RNA isolation kit (QIAGEN). If mammalian tissue is used as the source of RNA, a commercial reagent such as TRIzol Reagent (GIBCOL Life Technologies). A second cleanup after the ethanol precipitation step in the TRIzol extraction using Rneasy total RNA isolation kit may be beneficial.

Hot phenol protocol described by Schmitt, et al., (1990) Nucleic Acid Res., 18:3091-3092 is useful for isolating total RNA for yeast cells.

Good quality mRNA may be obtained by, for example, first isolating total RNA and then isolating the mRNA from the total RNA using Oligotex mRNA kit (QIAGEN).

Total RNA from prokaryotes, such as E. coli. Cells, may be obtained by following the protocol for MasterPure complete DNA/RNA purification kit from Epicentre Technologies (Madison, Wis.).

Before hybridization, the RNA samples may be fragmented. One preferred method for fragmentation employs Rnase free RNA fragmentation buffer (200 mM tris-acetate, pH 8.1, 500 mM potassium acetate, 150 mM magnesium acetate). Approximately 20 μg of RNA is mixed with 8 μL of the fragmentation buffer. RNase free water is added to make the volume to 40 μL. The mixture may be incubated at 94° C. for 35 minutes and chilled in ice.

IV. Design and Fabrication of Primer Array

a) Array Design

Preferred embodiments of the methods of the invention employ a plurality of probes on a substrate. The probes are suitable to be used as primers for template driven primer extension. The RNAs are used as templates for the primer extension reaction.

In one particularly preferred embodiment, oligonucleotide probes are synthesized or immobilized on a substrate in a 5′->3′ direction.

In one particularly preferred embodiment, high density oligonucleotide probe arrays are synthesized in a 5′->3′ fashion by using four 3′ MeNPOC-nucleoside-5′-phosphoramidites and photolithographic combinatorial synthesis methods disclosed in, e.g., U.S. Pat. Nos. 5,753,788, 5,744,101, and U.S. patent application Ser. No. 09/490,580, all incorporated herein by reference for all purposes.

The probes may be selected to detect a large number of, preferably more than 20, more preferably more that 100, even more preferably more than 1000 and most preferably more than 5000, transcripts. It is preferred to select more than one probe, such as more than 3, 5, 10, 15, 20, 25, 30, 35, 40 probes for each transcript. Probes used to detect one transcript is often grouped as a probe set. Methods for selecting optimal probes for gene expression are disclosed in for example, U.S. Pat. Nos. 5,800,992, and 6,040,138, U.S. patent application Ser. No. 60/252,808, filed Nov. 22, 2000, and U.S. patent application Ser. No. 60/252,617, filed Nov. 21, 2000, all incorporated here by reference for all purposes. The probes selected for hybridization assays are generally useful for primer extension as well.

In preferred embodiments, a consensus sequence of a transcript is constructed based upon information about the sequence of the transcript. Possible probes are generated. In some instance, sequence information in certain regions of a transcript may be ambiguous. In such instances, potential probes covering the ambiguous region may be eliminated. The probes may be selected for their hybridization according to certain rules (see, e.g., U.S. Pat. Nos. 5,800,992 and 6,040,138, both incorporated previously by reference in their entireties). In preferred embodiments, the probes are selected for their hybridization behavior according to their sequences (U.S. patent application Ser. No. 09/718,295, filed Nov. 21, 2000, previously incorporated by reference).

In addition to probes (primers) that are designed to be complementary with target sequences (perfect match probes, PMs), in some embodiments, the arrays may contain mismatch probes (MMs) that are designed to contain one or more mismatch bases. Normalization control and expression level control probes may also be included in the arrays (For a general discussion of the control probes, see, e.g., U.S. Pat. No. 6,040,138, which is incorporated herein by reference).

b) Fabrication of High Density Oligonucleotide Probe Arrays in5′-3′ Direction

Methods of forming high density arrays of oligonucleotides, peptides and other polymer sequences with a minimal number of synthetic steps are disclosed in, for example, 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes.

In brief, the light-directed combinatorial synthesis of oligonucleotide arrays on a glass surface proceeds using automated phosphoramidite chemistry and chip masking or optical direct write techniques. In one specific implementation, a glass surface is derivatized with a silane reagent containing a functional group, e.g. a hydroxyl or amine group blocked by a photolabile protecting group. Photolysis through a photolithogaphic mask or micromirror arrays is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

Because reverse transcriptases polymerize in 5′-3′ direction, in some embodiments, the oligonucleotides must be immobilized on a substrate in 5′-3′ direction. U.S. patent application Ser. No. 09/490,580, which is incorporated herein by reference for all purposes, disclosed methods for synthesizing oligonucleotide probes on a substrate in 5′-3′ direction.

In addition to photo-directed synthesis, other methods may also be employed for the fabrication of arrays with immobilized primers. For example, oligonucleotide synthesis may be conducted by selective delivery of reagents to specific locations using mechanic channels or ink-jet printers.

V. Hybridization

Nucleic acid hybridization simply involves contacting a probe and target nucleic acid under conditions where the probe and its complementary target can form stable hybrid duplexes through complementary base pairing.

It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency in this case in 6×SSPE-T at 37° C. (0.005% Triton X-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.). In one particularly preferred embodiment, hybridization is performed under high stringency, preferably at 1×MES at 45° C., more preferably 0.1×MES at 45° C.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Altering the thermal stability (Tₘ) of the duplex formed between the target and the probe using, e.g., known oligonucleotide analogues allows for optimization of duplex stability and mismatch discrimination. One useful aspect of altering the Tₘ arises from the fact that adenine-thymine (A-T) duplexes have a lower Tₘ than guanine-cytosine (G-C) duplexes, due in part to the fact that the A-T duplexes have 2 hydrogen bonds per base-pair, while the G-C duplexes have 3 hydrogen bonds per base pair. In heterogeneous oligonucleotide arrays in which there is a non-uniform distribution of bases, it is not generally possible to optimize hybridization for each oligonucleotide probe simultaneously. Thus, in some embodiments, it is desirable to selectively destabilize G-C duplexes and/or to increase the stability of A-T duplexes. This can be accomplished, e.g., by substituting guanine residues in the probes of an array which form G-C duplexes with hypoxanthine, or by substituting adenine residues in probes which form A-T duplexes with 2,6 diaminopurine or by using the salt tetramethyl ammonium chloride (TMACl) in place of NaCl.

Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)).

VI. Primer Extension

In particularly preferred embodiments, the hybridized RNAs are reverse transcribed with a reverse transcriptase to form a single stranded DNAs using the RNAs as templates. The 5′-3′ probe arrays, however, can also be used for primer extension using DNA, such as cDNA or Genomic DNA as templates.

Methods for performing in vitro reverse transcription reaction and appropriate conditions for such reactions are well known to those skilled in the art. Many source of reverse transcriptase may be used for the methods of the invention.

One key reagent for an in vitro reverse transcription reaction is the reverse transcriptase. Reverse Transcriptase is a DNA polymerase that synthesizes a complementary DNA strand from single-stranded RNA, DNA, or an RNA-DNA hybrid as a template. Reverse transcriptases derived from sources such the retroviruses avian myeloblastosis virus (AMV), Moloney murine leukemia virus (MMLV), Rous-associated virus type 2 or human immunodeficiency virus (HIV) are available from many commercial vendors including QIAGEN, PanVera, GENAXIS™ Biotechnology and Stratagene. Commercial vendors of reverse transcriptase typically provide instructions for conducting reverse transcription under optimal conditions. Other necessary reagents for such reactions, such as reaction buffer, are often available from those vendors.

In some embodiments, thermostable reverse transcriptase are used. Thermostable reverse transcriptase, such as the displayTHERMO-RT (available from PGC Scientifics Corporation) has two characteristics that greatly increase cDNA yield: it has no RNase H activity which minimizes cDNA primer degradation and it is thermostable so cDNA synthesis reactions can be run at temperatures high enough to melt RNA secondary structure. In preferred embodiments employing thermostable reverse transcriptase, the reaction is kept at a lower temperature until the reverse transcriptase extends from probes (primers) enough to make a stable RNA/cDNA hybrid. After this short period of extension, the temperature is increased to dissolve RNA secondary structure.

VII. Signal Detection and Data Analysis

In a preferred embodiment, the cDNA synthesized by extending immobilized probes is detected by labels. The labels may be incorporated by any of a number of means well known to those of skill in the art. However, in a preferred embodiment, the label is simultaneously incorporated during the extension reaction.

After the extension reaction and prior to the detection of extension products, the substrate may be washed under appropriate conditions to remove any nucleic acids that are not bound to the substrate. Because that it is not necessary to maintain the RNA/DNA duplex for signal to be detected, a high stringency washing may be used in some instances.

In a preferred embodiment, transcription amplification, a labeled nucleotide (e.g. fluorescein-labeled dATP and/or dCTP) incorporates a label into the reverse transcribed cDNA. In one particularly preferred embodiment, the primer extension products are labeled with a mixture of dNTP with at least one ddNTP, such as ddATP. The nucleotides may also be labeled with biotin.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. One particularly preferred method uses colloidal gold label that can be detected by measuring scattered light.

The label may be added to the target (sample) nucleic acid(s) during, or after the extension. So called “direct labels” are detectable labels that are directly attached to or incorporated into the target (sample) nucleic acid during the extension. In contrast, so called “indirect labels” are joined to the extension product. Often, the indirect label is attached to a binding moiety that has been attached to the extension products during the extension. Thus, for example, the extension product may be biotinylated during extension. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above.

In a preferred embodiment, however, the extension products are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The confocal microscope may be automated with a computer-controlled stage to automatically scan the entire high density array. Similarly, the microscope may be equipped with a phototransducer (e.g., a photomultiplier, a solid state array, a CCD camera, etc.) attached to an automated data acquisition system to automatically record the fluorescence signal produced by hybridization to each oligonucleotide probe on the array. Such automated systems are described at length in U.S. Pat. No. 5,143,854, PCT Application 20 92/10092, and U.S. application Ser. No. 08/195,889 filed on Feb. 10, 1994. Use of laser illumination in conjunction with automated confocal microscopy for signal detection permits detection at a resolution of better than about 100 μm, more preferably better than about 50 μm, and even more preferably better than about 25 μm, and most preferably better than 2 μm.

Scanners suitable for use with the embodiments of the invention are commercially available from, e.g., Affymetrix, Inc., Santa Clara, Calif., USA.

One of skill in the art will appreciate that methods for evaluating the hybridization results vary with the nature of the specific probe nucleic acids used as well as the controls provided. In the simplest embodiment, simple quantification of the fluorescence intensity for each probe is determined. This is accomplished simply by measuring probe signal strength at each location (representing a different probe) on the high density array (e.g., where the label is a fluorescent label, detection of the amount of florescence (intensity) produced by a fixed excitation illumination at each location on the array).

The fluorescence intensity data (or other signals) detected may be processed as described for gene expression monitoring without extension reaction. Some of the data processing methods are described in, e.g., U.S. Pat. Nos. 6,040,138 and 5,800,992, U.S. patent application Ser. Nos. 09/528,414, ______, attorney docket number 3357.1, ______, attorney docket number 3298.1, ______, attorney docket number 3309, ______, attorney docket number 3364, and ______, attorney docket number 3369.1, all incorporated herein in their entireties by reference for all purposes.

VII. Additional Applications

The methods of the invention have extensive practical applications, such as drug target identification, evaluation of toxicity, diagnostics and environment monitoring.

As described above, the methods of the invention may be used to monitor the expression of a large number of genes. The methods are particularly suitable for quantifying different forms of transcripts of a gene, such as alternative splicing products. Methods for identifying exon arrangements in transcripts are disclosed in, e.g., U.S. patent application Ser. No. 09/697,877, filed Oct. 26, 2000, incorporated herein by reference in its entirety for all purposes. The methods for probe design are particularly suitable for some embodiments of the invention for determining the arrangement of exons in transcripts, because the methods of the invention are less affected by 3′ bias. In embodiments for detecting arrangement of exons, probe (primers) are selected to tile the region bordering the exons. The tiling methods disclosed in U.S. patent application Ser. No. 09/697,877 and other applications/patent previously incorporated by reference are also useful for detecting sequence variation including mutations and polymorphisms including SNPs.

In addition, the method of the invention are very useful for transcription mapping. Methods for transcription mapping are disclosed in, e.g., U.S. patent application Ser. No. 09/641,081, which is incorporated herein in its entirety by reference for all purposes. In embodiments of the transcriptional mapping/annotation of the invention, probe (primers) are selected based upon genomic sequence and distributed along a region of interest in the genomic sequence. A region may be identified as being transcribed if transcripts are identified with the probe(s) derived from the region. Because of the methods of the invention is less vulnerable to 3′ bias, the preferred methods of the invention will result in more complete transcriptional mapping.

IX. Examples

The following examples illustrate the embodiments of the invention. Examples 1 shows the sucessful fabrication of high density oligonucleotide probe array and phosphoramidite monomers for the fabrication. Example 2 shows gene expression monitoring using primer extension.

a) Example 1. Fabrication of 5′-3′ High Density Oligonucleotide Probe Array

The phosphoramidite monomers may be prepared using the 3 step protocol.

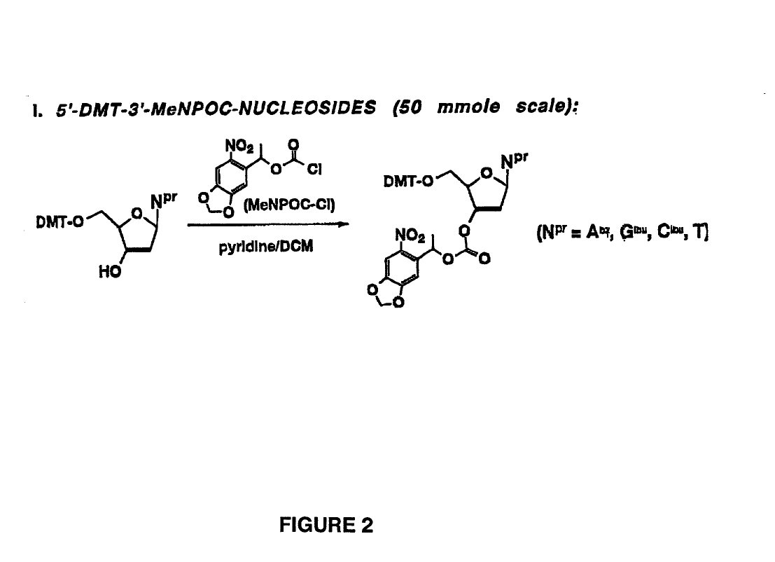

1) Synthesis of 5′-DMT-3′-MeNPOC-Nucleosides (50 mmole scale, see, FIG. 2)

The 5′ base protected nucleoside (50 nmole) is dried by co-evaporating three times with 150 ml anhydrous pyridine. The nucleoside is then dissolved or suspended in 150 ml of an anhydrous mixture of pyridine and DCM (2:1 by vol.) under argon, and cooled to −40° C. (dry ice-acetonitrile). A solution of 15 g (55 mmole) MeNPOC—Cl in 40 ml dry DCM is then added dropwise with stirring. After 30 minutes, the cold bath is removed, and the solution allowed to stir overnight at room temperature (TLC: DCM/EtOAc). After removing the solvents, the crude material is taken up in EtOAc and washed with water and brine. The organic phase is dried over Na₂SO₄ and evaporated to obtain a yellow foam.

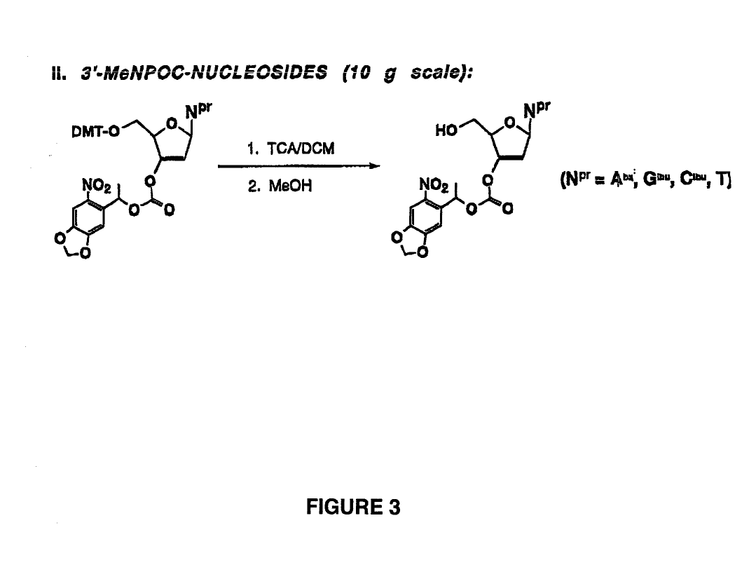

2) 3′-MeNPOC-Nucleosides (10 g scale, see FIG. 3)

The crude 5′-DMT-3′-MeNPOC-nucleoside (from 50 mmole reaction) is detritylated by stirring in 800 ml of 3% trichloroacetic acid/DCM plus 150 ml methanol at ambient temperature. The reaction is monitored by TLC (˜1:1 DCM-EtOAc). When detritylation is complete (˜60˜120 min.), the mixture is transferred to a separatory funnel and washed twice with 250 ml saturated aqueous NaHCO₃ (CO₂ is evolved) and then once with saturated NaCl. The organic phase is dried over Na₂SO₄ and evaporated to dryness. The residue is dried by twice adding and evaporating dry acetonitrile and then purified by flash chromatography on a 9.0(W)×12(H) column of silica gel (Merck 60A/flash) eluted with EtOAc+acetone (increasing from 0 to ˜50% by volume). Pure product (≧95% by HPLC) is obtained in ˜65% overall yield.

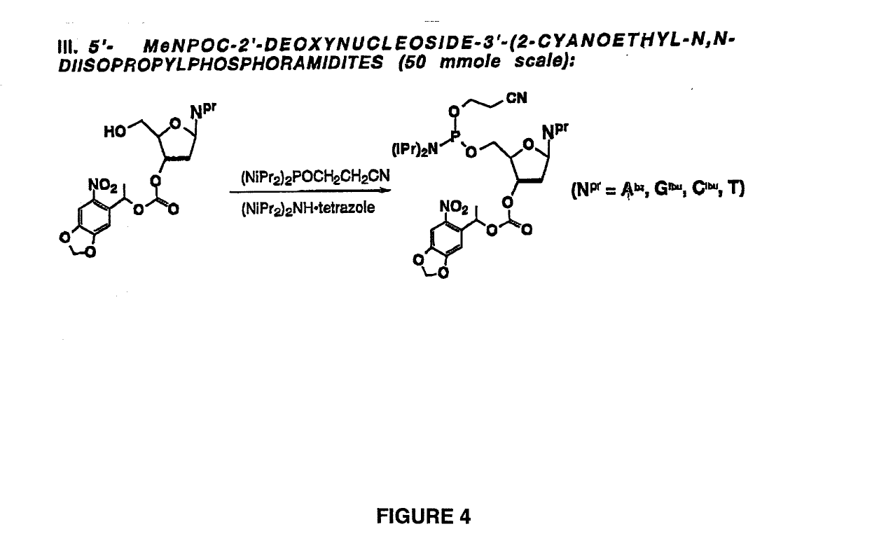

3) 5′-MeNPOC-2′-Deoxynucleoside-3′-(2-Cyanoethyl-N,N-Diisopropylphosphoramidites (50 nmol scale, FIG. 4).

The base protected 3′-MeNPOC-deoxynucleosides were phosphitylated using 2-cyanoethyl-N,N,N′,N′-3 tetraisopropylphosphorodiamidite. The following is a typical procedure (50 mmol scale):

2-cyanoethol- N,N,N′,N′-tetraisopropylphosphorodiamidite (16.6 g; 17.4 ml; 55 mmole) is added to a solution containing 50 mmole 5′-MeNPOC-deoxynucleoside and 4.3 g (25 mmole) diisopropylammonium tetrazolide in 250 ml dry CH₂CI₂ under argon at ambient temperature. Stirring is continued for 4-16 hours (reaction monitored by TLC: 10:45:45 EtcN-hexane-(CH2Cl2 or EtOAc). The organic phase is washed with 10% aqueous NaHCO3 and saturated brine, then drived over Na2SO4 and evaporated to drynes (yellow foam).

The crude phosphoramidites are purified by flash chromatrography on a short (6×6 cm) silica gel column (Merck 60A/flash, pre-conditioned & packed in 10% TEA-acetonitrile, then washed with DCM prior to loading the amidite), using the following elution gradients:

A(bz): DCM/0-20% EtOAc (+0.5% TEA)

C(ibu): hexane/50-100% EtOAc (+0.5% TEA)

T: hexane/50-100% EtOAc (+0.5% TEA)

G(ibu): 100% EtOAc

Fractions were checked first by TLC, then by HPLC to identify those with a minimum purity of 95%, which were combined and evaporated. The product is then dried by co-evaporating once the anhydrous acetonitrile and placing under high vacuum for 18-24 hours. Yields are about 80%, with a minimum purity of 95% as determined by ³¹P-NMR and HPLC (column:4 mm×250 MM 5 μM C18-silica; flow rate: 1 ml/min; solvent: 0.1M TEAA, pH 7.2/40-100T acetonitrile over 15 min.).

Two small test arrays were prepared for comparison, one using standard 5′ MeNPOC-nucleoside-3′-phosphoramidites (3′ probe attachment), and the other, 3′-MeNPOC-nucleoside-5′-phosphoramidites (5′-probe attachment). Otherwise, the arrangement of probe sequences in both arrays was identical.

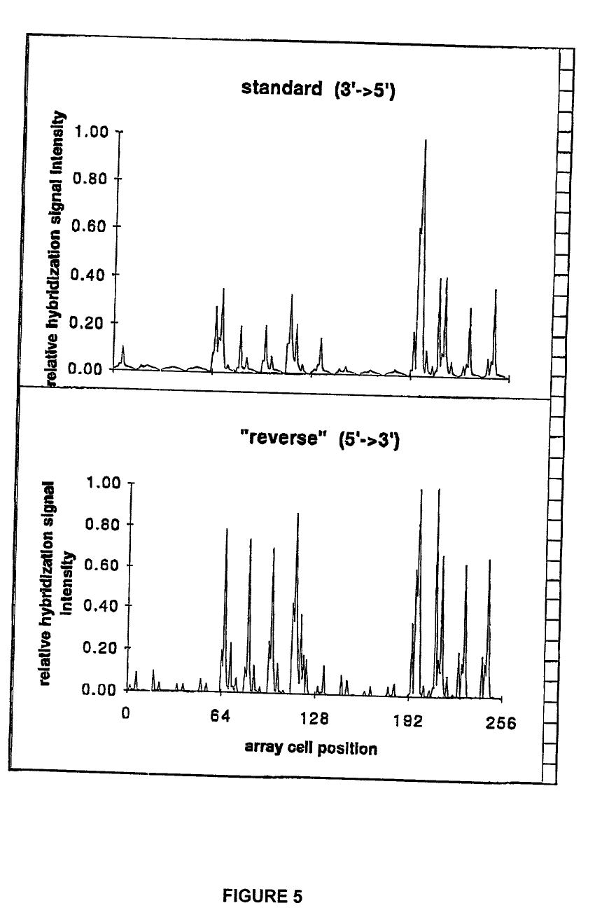

The arrays were exposed to a 10 nM solution of a target oligonucleotide (5′-flurescein labeled) in 5×SSPE for 4 hours, washed once with 5×SSPE, 7 imaged on a scanner. FIG. 5 shows a probe intensity vs. probe position for the two arrays. The two arrays displayed a close correspondence in terms of their hybridization behavior. Photolysis rate and coupling efficiencies observed with the 3′ MeNPOC building blocks are essentially identical to those obtained with the 5′-MeNPOC amidites.

b) Example 2. Reverse Transcription Using Primers on an Array

High density probe arrays were fabricated using the protocol in Example 1. The arrays have the same probe sequence as commercially available HuGenFL probe arrays (Affymetrix, Santa Clara, Calif.). However, the probes were synthesized in the 5′-3′ direction.

1) Materials and Methods

Sample preparation. BioB, BioC, BioD, Cre, Phe, Lys, and Thr genes were engineered so that trancribed RNAs contain poly (A) tails. The RNAs were used as spikes or control samples for hybridization. Final concentration on a probe array for the spikes were 0.58, 0.55, 0.42, 0.35, 0.26, 0.22, 0.18 nM for BioD, BioC, Lys, Cre, BioB, Phe, Thr, respectively.

Messenger RNA or total RNA were fragmented to 50-100 bases under the following conditions: 2 μl of 5×Affymetrix fragmentation buffer (200 mM Tris-acetate, pH 8.1, 500 mM KOAC, 150 mM MgOAC) is added to 8 μl of 10 ug of RNA. Incubation took place at 94° C. for 20 minutes.

First prehybridization. The chip was pre-wet with 1×MES solution and hybridized with 1×MES including 10 μM dNTPs, 0.5 mg/ml acetylated BSA and 0.1 mg/ml herring sperm DNA for 10 minutes at 45° C., followed by hybridizing the fragmented RNA for 16 hours at 45° C.

First wash. Chip was washed with fluidic station with 10 cycles of wash buffer A at 25° C., followed by 0.1×MES at 40° C. for 30 minutes.

Second prehybridization. The chip was hybridized with 1×MES including 10 uM dNTPs, 0.5 mg/ml acetylated BSA, 1 μg/μl tRNA and 0.1 mg/ml herring sperm DNA for 30 minutes at 45° C.

Reverse transcription. A 200 ul of cocktail of: 10 μl of each biotin-ddATP, dTTP, dCTP and dGTP, 10 μl of 10 mg/ml of BSA, 60 μl of 533 first strand buffer, 5 μl of RNasin, 5 μl of RNAguard, 10 ul of 10 mM DTT, 67.5 μl of DEPC H2O and 2.5 μl of superscript reverse transcriptase was into an array. The array was placed on a rotisserie in an oven at 45° C. for 30 minutes.

Second wash and scan. Briefly, an array was washed with Affymetrix fluidic station with 10 cycles of wash buffer A at 25° C., followed by 0.1×MES at 40° C. for 30 minutes. Then the array was stained for 30 minutes in SAPE solution at 25° C. 10 cycles of wash buffer A was applied again. The arrays were scanned with Affymetrix GeneArray® Scanner. Intensity data were analyzed using Affymetrix Microarray Suite or GeneChip® Analysis Suite.

2)Result

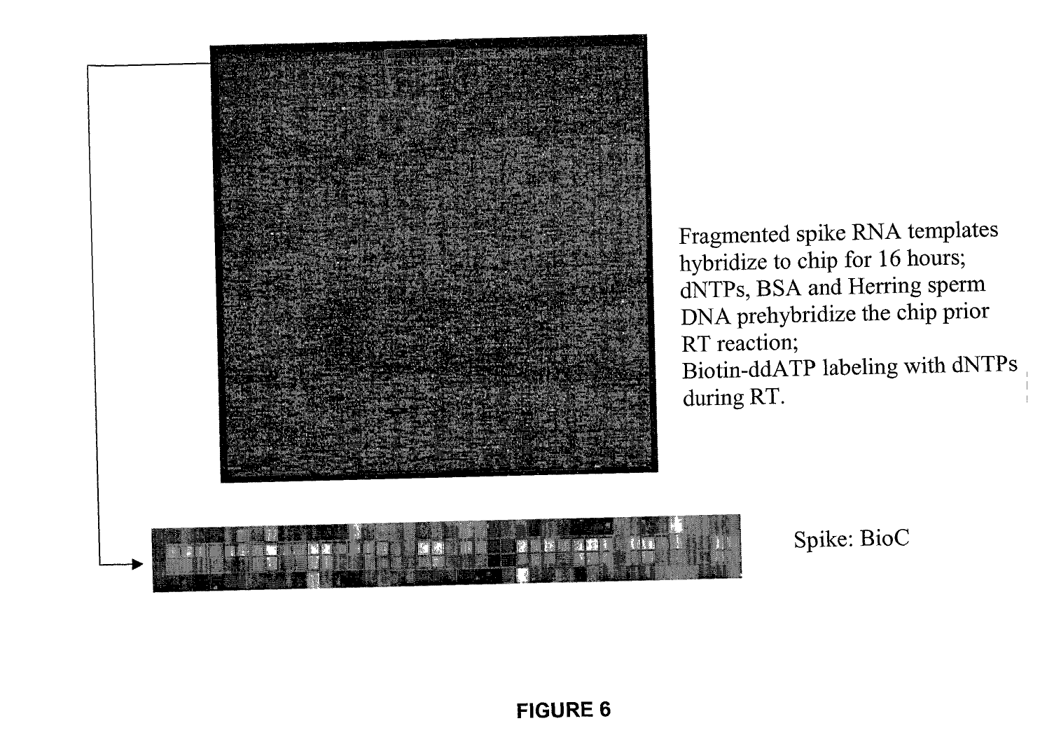

FIG. 6 shows an image obtained after reverse transcription reactions. The high lighted portion shows a probe set that was used to detect the bacteria control gene BioC. Table 1 shows the detection of spiked RNAs in a typical experiment. For genes that were not spiked, 2.20% of the genes were called present. In contrast, 100% of the spiked genes were called present in this experiment.

TABLE 1
Detection of Spiked RNAs

| Treament | tRNA blocked, PMT 660 |
|---|---|
| Present call | 2.20% |

| intensity | Avg Diff | Abs Call |
|---|---|---|
| AFFX-BioB-5_st | 715.8 | P |
| AFFX-BioB-M_st | 1236.7 | P |
| AFFX-BioB-3_st | 1029.8 | P |
| AFFX-BioC-5_st | 1074.5 | P |
| AFFX-BioC-3_st | 823.2 | P |
| AFFX-BioDn-5_st | 475.6 | P |
| AFFX-BioDn-3_st | 1165.8 | P |
| AFFX-CreX-5_st | 492.2 | P |
| AFFX-CreX-3_st | 758.2 | P |
| AFFX-LysX-5_at | 755.1 | P |
| AFFX-LysX-M_at | 678 | P |
| AFFX-LysX-3_at | 621.2 | P |
| AFFX-PheX-5_at | 208.2 | P |
| AFFX-PheX-M_at | 129.3 | P |
| AFFX-PheX-3_at | 307.9 | P |
| AFFX-ThrX-5_at | 428.4 | P |
| AFFX-ThrX-M_at | 637.9 | P |
| AFFX-ThrX-3_at | 881.7 | P |

### CONCLUSION

The present inventions provide methods for analyzing a large number of RNAs. It is to be understood that the above description is intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method for detecting a plurality of RNAs in a sample comprising:
hybridizing the sample with a substrate, wherein the substrate has a plurality of probes and wherein the probes are suitable for primer extension reactions;
synthesizing primer extension products with a nucleic acid polymerase, appropriate reagents and conditions, from the primers and using the RNAs as templates; and
detecting the primer extension products.

2. The method of claim 1 wherein the nucleic acid polymerase is a reverse transcriptase.

3. The method of claim 2 wherein the reverse transcriptase is a thermostable reverse transcriptase.

4. The method of claim 2 wherein the probes are oligonucleotide probes.

5. The method of claim 4 wherein the oligonucleotide probes are immobilized on the substrate in 5′-3′ direction.

6. The method of claim 5 wherein the oligonucleotide probes are synthesized on the substrated in 5′-3′ direction.

7. The method of claim 6 wherein the plurality of RNAs comprises at least 50 RNAs.

8. The method of claim 7 wherein the plurality of RNAs comprises at least 100 RNAs.

9. The method of claim 8 wherein the plurality of RNAs comprises at least 1000 RNAs.

10. The method of claim 9 wherein the plurality of RNAs comprises at least 5000 RNAs.

11. The method of claim 8 wherein each of the RNAs is targeted by at least 2 probes.

12. The method of claim 11 wherein each of the RNAs is targeted by at least 5 probes.

13. The method of claim 12 wherein each of the RNAs is targeted by at least 10 probes.

14. The method of claim 13 wherein each of the RNAs is targeted by at least 20 probes.

15. The method of claim 11 wherein the plurality of probes have at least 100 probes per cm² of the substrate.

16. The method of claim 15 wherein the plurality of probes have at least 1000 probes per cm² of the substrate.

17. The method of claim 16 wherein the plurality of probes have at least 10000 probes per cm² of the substrate

18. The method of claim 11 wherein the extension products are detected by using a label.

19. The method of claim 18 wherein the label is incorporated during the synthesizing.

20. The method of claim 18 wherein the label is attached to the extension products after the synthesizing.

21. The method of claim 19 wherein the reagent comprises at least one type of ddNTP.

22. The method of claim 21 wherein at least one type of ddNTP is labeled.

23. The method of claim 22 wherein the ddNTP is labeled with a biotin.

24. The method of claim 22 wherein the reagent comprises at least one type of dNTP.

25. The method of claim 24 wherein the reagent comprises dATP, dCTP, dGTP, and dTTP.

26. The method of claim 11 wherein the probes comprise tiling probes that are selected to tile regions of the RNA, and the method of claim 11 further comprising determining sequence variations by detecting the extension products of the tiling probes.

27. The method of claim 26 wherein the sequence variations are SNPs.

28. The method of claim 11 wherein the probes comprise tiling probes that are selected to tile the bordering regions of exons or putative exons, and the method of claim 11 further comprising determining the arrangement of exons in the RNAs by detecting the extension products of the tiling probes.

29. The method of claim 11 wherein the probes comprises probes that are designed to target subregions of a genomic sequence and the method of claim 11 further comprises determining whether the subregions of the genomic sequence is
